(19) United States
(12) Patent Application Publication
(10) Pub. No.: US 2020/0108170 A1
(43) Pub. Date: Apr. 9, 2020

(54) AUDITORY PROTECTION SYSTEM

(71) Applicant: Steve Mencanin, Aliso Viejo, CA (US)

(72) Inventor: Steve Mencanin, Aliso Viejo, CA (US)

(21) Appl. No.: 16/151,972

(22) Filed: Oct. 4, 2018

(51) Int. Cl. A61L 24/04; A61F 11/08

(52) U.S. Cl. CPC A61L 24/046; A61B 2017/00495; A61F 11/08

(57) ABSTRACT

The present invention relates to protection and communication apparatuses, methods and kits, especially for maintaining position and sealing of protective and/or communication headwear about a head/neck/ear region and in particular in maintaining position and sealing of an ear phone or hearing aid or the like within the acoustic meatus of an ear so as to exclude entry of sonic, sound or vibrational waves to the inner ear and tympanic membrane.

This application claims priority to copending US provisional application with the Ser. No. 62/567,857, which was filed Oct. 4, 2017

### FIELD OF THE INVENTION

The present invention relates to protection apparatuses, methods and kits, especially for maintaining position and sealing of protective headwear about a head/neck/ear region and in particular in maintaining position and sealing of an ear phone or hearing aid or the like within the acoustic meatus of an ear.

### BACKGROUND

It is known to wear removable headgear/helmets to protect the head from injury during contact or motor/biking sports. In a historical context, helmets were initially used as military protection and were intended to protect a combatant's head, face and neck from injuries inflicted by projective missiles such as spears and arrows. However, these protective headgears in whatever shape or form have suffered from the problem of movement about the head region when in position.

Safeguarding combatants in a modern battle zone remains a top priority. And the need is even more urgent than ever before because, unlike the spears and arrows of antiquity, bombs and bullets of the modem warfare are damaging to the combatants' hearing. The ear is the most sensitive organ in the body to the effects of blasts and so efforts continue to focus on designing more effective hear gear to protect the combatants' brains and ears from the harmful effects of loud blasts, and detect early signs of neurological and cognitive damage.

According to the Noise Pollution Clearinghouse, an anti-noise organization, a typical vacuum cleaner emits between 84 and 85 decibels. An M-16 rifle, on the other hand, registers at 157 decibels, and other weapons can be even louder. The explosion of roadside bombs, for example, is so powerful, it can rupture the eardrum and break bones inside the ear. The effects of blasts on the ear have been well documented with the effects on the inner ear being more frequent than in the middle ear. A temporary threshold shift of hearing acuity is common, often initially quite severe, but usually only transient Tinnitus is the most frequent reported ear symptom, and its recovery tends to parallel that of hearing loss. Balance problems are rare. Tympanic membrane perforations are common in those near to the explosion but they tend to close spontaneously in about 80% of cases.

Unfortunately, the full extent of hearing damage to troops may not be known for years as symptoms such as cognitive impairment or hearing loss may take time to fully manifest themselves. What is evident at present from the figures released by the Department of Veterans Affairs (DVA), is that of the 1.3 million US soldiers who served in Iraq since the war started in 2003, nearly 70,000 are currently collecting disability for tinnitus, and more than 58,000 are on disability for hearing loss, making hearing damage the number one disability produced by this war.

The danger of hearing loss in combat zones is not a new discovery, it has been known to be a leading disability among servicemen in World War II and Vietnam. In those battles, hearing was harmed by the artillery noise, bombing raids and tank battles. Yet, few lessons have been drawn from history: Early on in the Iraq war, the troops had been sent into combat without adequate hearing protection. A report published in 2006 in The ASHA Leader, a newspaper of the American Speech-Language-Hearing Association, states that there was an inadequate supply of earplugs for all soldiers, and a failure to provide unit commanders with information about troops having adequate hearing protection.

It is essential in a battle zone or conflict situation that effective and clear communication be maintained between personnel so a complete sound lock would not be appropriate. It is known to protect soldiers' hearing with, for example with the Combat Arms Earplug, which blocks harmful noise whilst allowing the troops to hear normally including noises that might alert them to danger and allow them to communicate with one another. The plugs are inserted into the ear and are held in either by expansion of the reliantly deformable rings and/or by an extension arm which curves around the pinna or outer ear. In addition, some troops have also received a state-of-the-art headset system, known as QuietPro. It contains digital processors that block out damaging sound waves from gunshots and explosions and still allow users to hear every day noises and communicate with one another. It will be appreciated that the QuietPro is not only an expensive piece of kit but is also bulky and would take up substantial space within a kit bag.

Prior art systems suffer from the disadvantage that the ear piece or helmet may be dislodged, lost or move during physical activity encountered in a conflict situation and so do not provide a fully reliable auditory protection system as sound waves can penetrate through gaps and spaces between an ear and its plug. A yet further disadvantage of prior art systems is the lack of bespoke fitment within the ear. It should be appreciated that the dimensions of the ear, and the acoustic meateus are non-uniform. Indeed, the ear shape is as individual as a fingerprint and ear prints have been used forensically to identify specific individuals in criminal scenarios. Therefore a fit one all system will not actually be fit for all purposes.

### BRIEF SUMMARY OF THE DISCLOSURE

An object of the present invention is to provide a robust and reliable auditory protection system.

A further object of the present invention is to provide an inexpensive, rapid and simple sealing system that can be used with existing protective products.

A yet further object of the invention is to provide a sealing system that is suitable for each individual despite the variation in ear shape.

In accordance with the present invention there is provided use of a polymerizable composition for sealing a substrate surface of a protective apparatus in situ to skin of a head, neck or ear region of an individual so as to provide a seal that prevents penetration or entry of acoustic waves to said individual, the seal being removable and formed in situ so as to contour a specific region of skin to which the substrate surface of the protective apparatus is applied.

Preferably, polymerizable composition polymerizes within 2 minutes of contact with the skin and/or substrate surface.

Preferably, the polymerizable composition comprises a silicone mixture.

Preferably, the silicone mixture comprises any one or more of the following components selected from the group comprising a siloxane polymer, a fumed amorphous silica, a platinum catalyst, a methyl hydrogen crosslinker, an inhibitor, a thixotropic agent, optionally wherein the thixotropic agent is polydimethyl siloxane and an adhesion promoter optionally wherein the adhesion promoter is tetrapropoxysilane.

Preferably, the silicone mixture comprises:

(i) a first silicone elastomer component comprising a first platinum catalyst; and
(ii) a second silicone elastomer component comprising a silicone crosslinker.

Preferably, the silcone mixture has a work time of between 30-90 seconds, and a cure time of between 1-5 minutes.

Preferably, the seal has a hardness of between 0 on the Shore 00 durometer scale and 40 on the Shore A durometer scale.

Preferably, the seal has a hardness of between 15-25 on the Shore A durometer scale.

Preferably, the seal has an elongation at break of between 200-800%. Preferably, the seal has an elongation at break of between 400-650%. Preferably, the seal has a tensile strength of between 100-2000 psi.

Preferably, the seal has a tensile strength of between 200-800 psi.

Preferably, wherein prior to application of the polymerizable composition there is provided use of a base component in order to increase adhesion and/or improve cure time of the polymerizable composition.

Preferably, the base component is applied as a spray, foam or wipe.

Preferably, the base component comprises a catalyst that accelerates polymerization of the polymerizable composition.

According to a further aspect of the invention there is provided a removable elastic seal comprising a polymerized composition, the seal being sized and shaped to naturally mimic contours of a head circumference, neck or the acoustic meatus of an ear of a protective apparatus device and to maintain position of said device, the seal comprising:

(i) a first silicone elastomer component comprising a first platinum catalyst; and
(ii) a second silicone elastomer component comprising a silicone crosslinker. Preferably, the seal includes any one or features hereinbefore described.

According to a further aspect of the invention there is provided a method of treatment of an inner ear injury, comprising:

applying to at least one of an auricle and an external ear canal a polymerizable composition;

wherein the polymerizable composition is formulated to cure to a hardness within 5 minutes; and

allowing the polymerizable composition to harden in the at least one of the auricle and external ear canal to the hardness, wherein the hardness is between 0 on the Shore 00 durometer scale and 40 on the Shore A durometer scale to so form an elastic protective barrier.

The method advantageously prevents auditory damage to an individual, by providing a removable, elastic seal about a head/neck or ear region and a protective apparatus so as to exclude or prevent entry of sonic, sound or vibrational waves to the inner ear and/or tympanic membrane.

According to a yet further aspect of the invention there is provided a method of protecting an inner ear from barotrauma, acoustic trauma, vestibular impairment, and noise induced hearing loss, comprising:

applying to at least one of an auricle and an external ear canal a polymerizable composition;

wherein the polymerizable composition is formulated to cure to a hardness within 5 minutes; and

allowing the polymerizable composition to harden in the at least one of the auricle and external ear canal to the hardness, wherein the hardness is between 0 on the Shore 00 durometer scale and 40 on the Shore A durometer scale to so form an elastic protective barrier.

Preferably, the methods include any one or features hereinbefore described.

According to a yet further aspect of the invention there is provided a device comprising a piece of protective head/neck ware for positioning about a head or an ear piece to be placed into an ear, an outer or peripheral substrate of said device having a coating of a polymerizable composition. The composition having any one or more of the features hereinbefore described. The polymerizerable composition having being applied to the peripheral region of the substrate of the device.

According to a yet further aspect of the invention there is provided a kit for preventing auditory damage during blast situations, comprising:

(i) a base component comprising at least one of an adhesion promoter, a catalyst that accelerates polymerization of the polymerizable composition;
(ii) a polymerizable composition formulated to polymerize to form a removable seal; and optionally
(iii) at least one ear plug.

Preferably, the kit includes any one or features hereinbefore described.

### DETAILED DESCRIPTION

The inventive subject matter provides compositions and methods for forming a secure seal about a head/neck/ear to exclude soundwave that could cause ear damage using a composition that polymerizes in situ within and/or around an earpiece (e.g., sound attenuating ear piece or ear piece of a communication device that acts as an in-ear speaker and/or microphone) or other protective headwear that is bespoke to the individual. Such seal can be formed from any polymerizable or cross linkable composition to generate a barrier on the substrate it is attached to, especially skin, on the head/neck ear region. Contemplated seals can act as a barrier to completely or substantially completely block the substrate from exposure to the environment (e.g., sound, water, dirt, sun/radiation exposure, etc. Viewed from a different perspective, it is contemplated that a seal formed on at least head/neck/ear skin can be configured to remain substantially adhered to the substrate, that being a surface of the equipment (e.g., at least 80% of the seal remains adhered to the skin, at least 90% of the seal remains adhered to the skin, at least 95% of the seal remains adhered to the skin, 100% of the seal remains adhered to the skin) for a period of at least one hour e.g., at least 2 hours, at least 3 hours, at least 6 hours), even under water, or a period of at least 1 day (e.g., at least 2 days, at least 3 days, at least 4 days, at least 5 days). Thus, seals presented herein can act as a temporary adhesive for communication devices, sensors (e.g., vital sign, temperature, motion, GPS, etc.), and other personal equipment that secure the item to a person's skin.

Furthermore, contemplated seals can be removed, typically intact, from the skin without injury or epithelial cell removal. It should therefore be appreciated that the seal can be partial or complete with respect to the site of attachment and can be a semi-permanent or temporary, removable seal. Viewed from a different perspective, it should be recognized that the seal can be formed over a surface of a head/neck/ear area by application of the film to the site of attachment and/or the periphery of the head/neck/ear apparatus, while the outer ear canal can be sealed with the compositions to protect the inner ear from injury and sound where the ear drum is perforated.

Accordingly, to summarize on the numerous advantages provided by the present invention, the compositions provide a seal for acoustic waves as well as a temporary strong adhesive that allows equipment to be secured in place. The molding effect of the compositions creates a fitting to the head/neck/ear geometry such that devices or equipment that would otherwise not be easily retained can now be securely worn, while allowing for manual removal.

With respect to suitable compounds and compositions it is contemplated that all commercially available and/or custom-formulated polymerizable and/or cross linkable compositions are for use herein, including vinyl-based cure systems, silicone-based cure systems, peroxide-cure systems, heat cure systems, room temperature vulcanizing moisture cure systems, temperature activated systems, photoinitiated cure systems, and addition-cure systems. Additionally, all suitable polymerization reactions are contemplated, including hydrosilylation polymerization, condensation polymerization, and addition (chain-growth) polymerization (e.g., photopolymerization), and radical polymerization. In this context it should be appreciated that cross linking and polymerization can be used interchangeably as both reactions increase the size of a polymeric backbone (and molecular weight) and decrease flowability to a point of cure.

In some preferred aspects, the polymerizable composition will result in a cured seal having sufficient elasticity to allow the user to move around comfortably and naturally. The polymerizable composition is typically odorless, non-toxic, hypoallergenic, compatible with other treatments, bacteriostatic, non-explosive, non-temperature sensitive, and removable as a single piece, or in sections, after curing.

The polymerizable composition can comprise a one part system, for example, where the polymerizable composition is temperature or light activated. Alternatively, the polymerizable composition can comprise a multi-part system, for example, where a catalyst and a cross-linking component must be kept separate prior to use to prevent premature and undesired curing.

One type of a preferred polymerizable composition comprises a two-part elastomer system that cures at room (20 degree Celsius) or body (37 degree Celsius) temperature and includes (a) a first formulation including a polymer and catalyst (e.g., silicone polymer and platinum or other suitable metal catalyst), and (b) a second formulation comprising a polymer and a crosslinker. One or both of the formulations could include one or more of a filler, a thixotropic agent, an adhesion promoter, and a cure inhibitor to control the cure kinetics.

Where the first and second formulations are separately packaged or contained in a dual chambered system (e.g., a dual chamber syringe that is hermetically closed, or dual chamber flexible pouch with frangible seal between the chambers), cross linking cannot occur until the two components are mixed together (e.g., layered or applied as a mixture). In some contemplated embodiments, the polymer is a silicone polymer (e.g., siloxane polymer) with a polymer backbone of alternating silicone and oxygen atoms (i.e., siloxane bonds), and hydrocarbon (saturated, unsaturated, aromatic) organic side groups such as methyl, phenyl or vinyl, or a hydrogen attached to the silicon atoms. The siloxane polymer can comprise between 20-100 wt %, more preferably at least 50 wt %, and even more preferably at least 70 wt % (e.g., between 75-85 wt %, between 78-82 wt %) of the polymerizable composition (i.e., of the combined two part formulation where the catalyst and crosslinker are combined).

For example, where polydimethylsiloxane (PDMS) is used, it can be a linear polymer made up of repeating Si-0-Si linkages and a reactive vinyl group on both ends of the polymer chain. There may be organic side groups such as dimethyl bonded to every silicone molecule the backbone of the polymer. Siloxane polymers can also be substituted with diphenyl, methylphenyl, trifluoropropyl, or any combination thereof. Some exemplary siloxanes include oligosiloxanes, polydimethylsiloxane (PDMS), vinyl-endblocked polydiphenyl siloxane, vinyl-endblocked polymethylphenylsiloxane, vinyl-endblocked trifluoropropyl siloxane, vinyl-endblocked polydiethyl siloxane, trimethyl-endblocked methylvinyl polydimethylsiloxane, trimethyl-endblocked methylvinyl polydiphenylsiloxane, trimethyl-end blocked methylvinyl polymethylphenylsiloxane, trimethyl-endblocked methylvinyl polytrifluoropropylsiloxane, and trimethyl-endblocked ethylvinyl polydimethylsiloxane. Contemplated siloxanes can be optically clear, non-toxic and non-flammable.

All suitable chain lengths of the siloxane polymer are contemplated, including between 10-2,500 repeating units long, between 200-1,000 repeating units long, or between 200-400 repeating units long, or between 300-600 repeating units long, or between 300-400 repeating units long (e.g., 340-360), or between 500-800 repeating units long, or between 700-1,000 repeating units long. Thus, siloxane polymers may have an average molecular weight of between about 500-5,000 Daltons, or between about 5,000-20,000 Daltons, or between about 15,000-35,000 Daltons, or between about 35,000-55,000 Daltons, or between about 55,000-100,000 Daltons, or more.

According to another embodiment, a polymer can include a main chain formed primarily of organosiloxane units. Among the silicone compounds contemplated, some may display both curing and adhesive properties, for example depending on the proportion of silicone or whether they are used with a particular additive. It may therefore be possible to adjust the properties of said compositions according to the proposed use.

In some contemplated embodiments where the polymer is a siloxane, the crosslinker is a siloxane crosslinker such as a methyl-hydrogen crosslinker. The crosslinker can comprise between 0.1-50 wt %, between 0.1-10 wt %, and more preferably between 1-5 wt % (e.g., 2 wt %) of the polymerizable composition. An exemplary siloxane crosslinker used in some contemplated compositions is a small chain polymer that is trimethyl endblocked, making the ends of the chain non-functional. All suitable chain lengths of the crosslinker are contemplated, including for example, between 1-100 repeating units, more preferably between 1-50 units, and more preferably between 5-15 units (e.g., 10 units wherein the molecular weight is 800 Daltons).

Along the backbone of the crosslinker can be reactive methyl-hydrogen side groups which can comprise between 1-99 mole %, more preferably between 20-80 mole %, and more preferably between 40-60 mole % (e.g., 50 mole %) of the crosslinker. The remaining mole % can comprise dimethyl side groups. Where each of the methyl-hydrogen side groups and the dimethyl side groups make up approximately 50 mole %, approximately half of the repeating units of the crosslinker will be dimethyl, and approximately half will be methyl hydrogen. Other contemplated crosslinkers include hydride-endblocked polydimethylsiloxane, hydride-endblocked methylhydrogen polysiloxane, trimethyl-endblocked methylhydrogen methylvinyl polysiloxane, trimethyl-endblocked 100 mole % methylhydrogen polysiloxane, hydride-endblocked polydiphenylsiloxane, and hydride-endblocked phenylhydrogen polysiloxane. Although the exemplary crosslinkers described above are siloxane crosslinkers, it should be appreciated that a person skilled in the art would be able to select a suitable crosslinker based on the polymer included in the polymerizable compositions.

The catalysts of contemplated polymerizable formulations can comprise a peroxide, platinum, tin, a combination thereof, or other suitable catalyst. An exemplary platinum catalyst for hydrosilylation reactions can comprise a complex of platinum with a vinyl siloxane acting as a ligand. An example of this is the Karstedt's catalyst. Other contemplated catalysts include, rhodium complex in vinly silicone fluid, organotin catalyst such as dibutyltin dilaurate, stannous octoate, dibutlytin diacetate, peroxide catalysts such as benzoyl peroxide, 2,4 dichlorobenzoyl peroxide, dicumyl peroxide, 2,5-dimethyl-2,5-di(tert-butylperoxy)hexane. In still further contemplated aspects, platinum may be replaced at least to some degree by other metals, including silver and copper, in nanoparticulate form or ionic form, which advantageously will also provide antimicrobial effect.

The catalyst can be present in the formulation in any suitable amount, for example, between 0.001-10 wt % (of the combined two part formulation where the catalyst and crosslinker are combined), more preferably between 0.01 and 1 wt %, and more preferably between 0.07 and 0.13 wt % (e.g., 0.1 wt %) of the polymerizable composition, and can include between 1-250 ppm, between 5-70 ppm, more preferably between 15-60 ppm (e.g., 30 ppm) of pure platinum. Notably, the amount of catalyst will affect the working time of the mixed composition, and higher quantities of catalyst will decrease the time required to seal a bleed. Thus, higher than normal (e.g., as recommended by manufacturer or published in the relevant art) quantities of catalyst are generally preferred.

The platinum catalyst will preferably be separated from the crosslinker until placed at the attachment site. Alternatively or additionally, the platinum catalyst can be combined with the crosslinker no more than 5 minutes, no more than 3 minutes, and preferably no more than 1 minute or 0.5 minute prior to being placed on the periphery of the protective apparatus or skin at the site of attachment. Alternatively or additionally, the component or formulation comprising the platinum catalyst can be placed within the injury before or after the formulation or component comprising the crosslinker is placed on the periphery of the protective apparatus or skin at the site of attachment.

As discussed in more detail below, a base component (e.g., spray or wipe) comprising the same or different catalyst could be applied prior to any of the first formulation (including the platinum catalyst) and the second formulation (including the crosslinker). Therefore, contemporaneous application of the catalyst and the crosslinker by mixing the composition at the time of application is also contemplated.

Where a filler is included in the polymerizable formulation, an exemplary filler includes amorphous fumed silica having a surface area of between 100-300 m²/gram (e.g., approximately 200 m²/gram). Other contemplated fillers include fumed silica with low surface area (e.g., 100 m²/gram), fumed silica with high surface area (e.g., 400 m²/gram), precipitated silica, diatomaceous earth, titanium dioxide, zinc oxide, barium sulfate, colloidal silica, and boron nitride.

The filler can comprise between 0-80 wt %, more preferably between 5-35 wt % and even more preferably between 10-23 wt % (e.g., 16 wt %) of the combined two part formulation where the catalyst and crosslinker are combined. The surface of the silica can be treated with trimethyl silyl groups so that it is more soluble with the polymer.

A suitable thixotropic additive (e.g., a compound that reduces the flowability of a material rendering it non-slump) can also be included in some contemplated polymerizable compositions in any suitable amount. For example, the thixotrope can comprise between 0.1-5 wt %, between 0.5-2.5 wt %, and more preferably between 1-2 wt % (e.g., 1.5 wt %) of the combined two part formulation where the catalyst and crosslinker are combined. An exemplary thixotrope included in some contemplated formulations is a hydroxyl endblocked polydimethyl siloxane with a chain length of between 10-20 repeating units (e.g., 15 repeating units with a molecular weight of 1100 Daltons). The hydroxyl groups on the polymer ends can react with the surface hydroxyl groups of the fumed silica causing the silica to become less flowable.

Suitable adhesion promoters can also be included in the polymerizable composition to increase the bond strength of the adhesive (polymerizable composition or seal) to the substrate (e.g., periphery of appartus) as curing occurs. Tetrapropoxysilane is an exemplary adhesion promoter commonly used in silicone primers. The adhesion promoter, when included in the polymerizable composition, can comprise between 0.01-10 wt %, between 0.1 and 5 wt %, and more preferably between 0.4 and 1.2 wt % (e.g., 0.8 wt %) of the polymerizable composition.

Additional adhesion promoters suitable for contemplated polymerizable formulations include those shown in Table 1, along with relevant results. In the below example, equal parts of the first and second components as described in Table 2 below including the different adhesion promoters were mixed and a thin layer was applied to a forearm and allowed to polymerize at room temperature. The samples were evaluated by recording the time that the edges began to lift from the skin. Once the edges lifted, the samples were peeled off and evaluated qualitatively for how difficult it was to peel completely off the skin. Each adhesion promoter was evaluated, and the results are described in Table 1 below. All percentages used herein are weight percentages (wt %) unless otherwise indicated.

TABLE 1

| | Edge Lifting | |
|---|---|---|
| Formulation | Began | Adhesion to skin |
| Formulation of table 2 with no | 1 hour | poor |
| adhesion promoter | | |
| Formulation of table 2 with 1% | 5 hours | good |
| Tetrapropoxysilane | | |
| Formulation of table 2 with 1.5% | 5 hours | good |
| Tetrapropoxysilane | | |
| Formulation of table 2 with 1% | 3.25 hours | good |
| 3-aminopropropyltrimethoxysilane | | |
| Formulation of table 2 with 1.5% | 3.25 hours | good |
| 3-aminopropropyltrimethoxysilane | | |
| Formulation of table 2 with 1% | 2 hours | poor |
| Tris(2-methoxyethoxy)(vinyl)silane | | |
| Formulation of table 2 with 1.5% | 2.5 hours | poor |
| Tris(2-methoxyethoxy)(vinyl)silane | | |
| Formulation of table 2 with 1% | 2 hours | poor |
| Vinyltriethoxysilane | | |
| Formulation of table 2 with 1.5% | 2.5 hours | poor |
| Vinyltriethoxysilane | | |
| Formulation of table 2 with 1% | 4 hours | good |
| Tetrakis(2-methoxyethyl)ester | | |
| Formulation of table 2 with 1% | 4 hours | good |
| Tetrakis(2-methoxyethyl)ester | | |
| Formulation of table 2 with 1% | 5 hours | good |
| Trimethoxy-7-octenylsilane | | |
| Formulation of table 2 with 1% | 5 hours | good |
| Trimethoxy•7-octenylsilane | | |
| Formulation of table 2 with | 6.5 hours | good |
| 0.25% N-(triethoxysilylpropyl)- | | |
| 0-polyethylene oxide | | |
| urethane | | |
| Formulation of table 2 with | 6.5 hours | good |
| 0.5% N-(triethoxysilylpropyl)-0- | | |
| polyethylene oxide urethane | | |
| Formulation of table 2 with | 18 hours | Excellent(synergistic |
| 0.25% N-(triethoxysilylpropyl)- | | effect with |
| 0-polyethylene oxide | | respect to |
| urethane and 1.5% | | adhesion where |
| Tetrapropoxysilane | | two adhesion |
| | | promoters were |
| | | used) |

The curable formulations used in each of the compositions of Table 1 are shown in Table 2. The adhesion promoter(s) of Table 1 were added to Part 2 of the formulation. However, it should be appreciated that the adhesion promoter could alternatively or additionally be added to Part 1 of the formulation. It should also be appreciated that the percentages shown in Part 2 below are modified once the adhesion promoter(s) are added.

TABLE 2

| Part 1 | | Part 2 | |
|---|---|---|---|
| Component | wt % | Component | wt % |
| Vinyl endblocked polydimethyl | 79.95 ± 5 | Vinyl endblocked polydimethyl | 76.985 ± 5 |
| siloxane polymer | | siloxane polymer | |
| Fumed silica with surface area of | 19.99 ± 2 | Fumed silica with surface area of | 19.2 ± 2 |
| 200 m2/gram | | 200 m2/gram | |
| Platinum catalyst complex | 0.06 ± .3 | Trimethyl endblocked methyl- | 3.8 ± 1 |
| | | hydrogen siloxane polymer | |
| | | crosslinker (containing 50% | |
| | | methyl | |
| | | hydrogen and 50% dimethyl) | |
| | | 1,3,5,7- | 0.015 ± .005 |
| | | tetramethyl-1,3,5,7- | |
| | | tetravinyl-cyclotetrasil oxane | |

It is also contemplated that inert pigments can be suspended in the polymerizable formulations without leaching. Suitable uses for inert pigments can include visibility, aesthetics (e.g., with designs) or camouflaging (e.g., flesh tones, bright tones, or any other suitable tones), or color-coding (e.g., red for laceration with lodged foreign particles, yellow for infected wound, etc.). Some contemplated powdered pigments can advantageously be broken down to a size of less than 20 microns, more preferably less than 15 microns to allow for even distribution or dispersion throughout the polymerizable formulation. Additionally or alternatively, concentrated liquid or gum color pigments can be added to one or more components of the polymerizable formulation.

Radio opaque or other particles (e.g., barium sulfate, zirconium dioxide) could be suspended or otherwise incorporated into the polymerizable formulations such that the cured seal can be detected by X-ray, computed tomography scans, ultrasound imaging or MRI scans. In some preferred embodiment, at least 8 wt %, more preferably at least 10 wt % (e.g., at least 11 wt %, between 8-50 wt %, between 10-20 wt %) is included in the combined two part polymerizable formulation for detection by X-ray. The radio opaque particles could be added to the polymerizable formulation in any suitable matter, and could even be pre-mixed with one or more of its components. For example, the radio opaque particles could be mixed in with the first formulation component, second formulation component, silicone polymer, platinum catalyst, crosslinker, adhesion promoter, cure inhibitor, filler, thixotropic agent, or any combination thereof. Alternatively, the particles could be added as a top layer to the composition while curing.

It is contemplated that the first and second formulations can react with each other at various temperatures, including for example at temperatures between −20 and 80 degrees Celsius, more typically between 0 and 60 degrees Celsius, and even more typically between 10 and 50 degrees Celsius. For example, it is contemplated that the formulations will be capable of reacting together to form a seal at room temperature (20±5° C.) and atmospheric pressure, or advantageously in the presence of a catalyst, by a hydrosilylation reaction or a condensation reaction, or a crosslinking reaction in the presence of a peroxide.

A complete seal can be formed within 20 minutes, within 10 minutes, more preferably within five minutes, within three minutes, within two minutes, or even within one minute. The seal can have any suitable thickness to treat or manage the injury, for example a thickness of between 0.1 mm and 50 mm, between 1 mm and 40 mm, between 1 mm and 20 mm, between 1 mm and 10 mm, or between 1 mm and 5 mm.

Upon full curing, the seal can have a hardness sufficient to prevent unwanted flowing of the seal. For example, the seal can have a hardness of at least 1, or at least 5, or at least 10 on the Shore 00 durometer scale, at least 1, or at least 5, or at least 10 on the Shore A scale, a hardness of between 0 on the Shore 00 durometer scale and 40 on the Shore A durometer scale, a hardness of between 10 on the Shore 00 durometer scale and 30 on the Shore A durometer scale, a hardness of between 15-25 on the Shore A durometer scale, or a hardness of between 18-22 on the Shore A durometer scale. The work time of the polymerizable composition can be approximately half of the cure time (e.g., about sixty seconds where the cure time is about two minutes).

Additionally or alternatively, the seal can have an elasticity that allows for movement and stretching of the body without compromising the seal or causing discomfort or pain. For example, some contemplated compositions will have an elasticity at break of at least 200%, more preferably at least 300%, and more preferably at least 400% (e.g., between 200-1000%, between 400 and 800%, between 450-600%). As used herein, the term “% elasticity at break” refers to the extension of a length of a cured seal from an unstretched and normal configuration before tearing, at room temperature, wherein the cured seal has a thickness of between 3-5 mm in the unstretched, normal configuration. For example, where a cured seal has an at least 180% elasticity at break, the cured seal, when normally having a thickness of between 3-5 mm, can be stretched to at least 180% of its length before tearing (e.g., from 10 mm to at least 18 mm before tearing). Viewed from a different perspective, the seal can have a tensile strength that allows significant force to be applied while maintaining its integrity (e.g., between 100-2000 psi, between 200-800 psi, between 400-650 psi).

Compounds and compositions according to the inventive subject matter can also be used to affix non-tissue materials to a person such as the periphery of protective head/neck/ear wear, and particularly contemplated non-tissue materials include silicon sheds or gauze. Advantageously, as the compounds and compositions according to the inventive subject matter undergo polymerization silicon sheets are bonded to the compounds and compositions according to the inventive subject matter and may so further provide a protective cover or wrap.

Where desirable, contemplated compounds and compositions may also at least partially engulf foreign objects such as wires or the like, which can be removed after polymerization together with the cured materials. Additionally, it should be appreciated that the polymerizable materials presented herein allow for polymerization in the presence of water, sweat, blood, and other body fluids.

Additionally or alternatively, the base component could act as or include a primer that promotes adhesion of the polymerizable composition to the skin when applied, and include a catalyst to decrease the cure time at the attachment edges of the polymerizable composition. Preferably, the base component will not negatively impact the polymerizable composition's ability to adhere to the skin, or cure in situ in a short amount of time.

Contemplated adhesion promoters can include a silane coupling agent containing one or more functional groups that bond with the polymerizable composition or components thereof. Some contemplated adhesion promoters include a tetramethoxysilane, a tetraethoxysilane, a tetraisopropoxysilane, a tetrapropoxysilane, a tetrabutoxysilane, and a tetraacetoxysilane, a 3-aminopropropyltrimethoxysilane, tris(2-methoxyethoxy)(vinyl)silane, vinyltriethoxysilane, tetrakis(2-methoxyethyl)ester, and trimethoxy-7-octenylsilane.

When included, the adhesion promoter(s) can be present in the base component in any suitable amount. For example, it is contemplated that the adhesion promoter can be present in a spray, ointment, jelly or other composition in any suitable concentration, including for example, a concentration of between 0.1-100 mg/ml, between 0.1-75 mg/ml, between 0.1-50 mg/ml, between 0.1-10 mg/ml, or between 0.5-10 mg/ml.

Similarly to the polymerizable composition, the base component can also include a silicone or other catalyst that promotes curing of the polymerizable composition, which can be present in any suitable concentration (e.g., between 0.001-50 mg/ml).

Where the base component is a silicone primer, the primer will typically include one or more reactive silanes, a catalyst, and a solvent carrier (among other things). The reactive silanes can include a reactive group that is compatible with the polymerizable composition, and another reactive group that is compatible with the substrate (e.g., skin, tissue) to thereby promote adhesion of the polymerizable composition to the substrate. One exemplary silicone primer comprises between 88-93 wt % isopropyl alcohol (e.g., 88 wt %), between 1-5 wt % tetrapropoxy silane (e.g., 3%), between 1-5 wt % titanium IV butoxide (e.g., 3%), and between 0.01-2 wt % platinum catalyst (e.g., 1 wt %). However, all suitable silicone primer compositions are contemplated.

The PHOSITA should appreciate that different materials can be obtained from different commercial suppliers. For example, it is contemplated that components of some contemplated polymerizable compositions or base component can be obtained from commercial suppliers, for example, Silbond Corporation, Chemat, H.W. Sands Corp., Fluorochem USA, Gelest, Inc., Dupont Performance chemicals, Nusil Technology, Power Chemical Corporation, Rhodia Silicones, Reliance Silicones, or Zentek.

In still further contemplated uses, the compounds and compositions of the inventive subject matter may be used to temporarily glue down silicon and/or other compression strips/tapes and (silicone) tubing or electrical wires to the site of attachment of the combatant. Moreover, the compounds and compositions of the inventive subject matter may also be used to temporarily seal/close off the outer or inner ear canal or a nostril. Such sealing may be performed for the purpose of avoiding water or contaminant ingress into the ear canal, or to prevent sound from entering a damaged inner ear.

Contemplated polymerizable compositions, upon forming a seal in situ, will preferably be elastic or semi-elastic. For example, some contemplated compositions will have an elasticity at break of at least 200%, more preferably at least 300%, and more preferably at least 400% (e.g., between 200-1000%, between 400 and 800%, between 450-600%), to allow for movement and stretching of the body without compromising the seal. Likewise, contemplated compounds and compositions will be deformable upon compression (e.g., using moderate manual force or force common with wound compression upon motion of a body part having the wound). For example, some contemplated seals will have a hardness of at least 10 on the Shore 00 durometer scale, and more preferably at least 10 on the Shore A scale.

Most notably, upon curing, contemplated compounds and compositions were not only sufficiently flexible to remain at the site attachment, but could be removed intact from the site.

Preferably, the polymerizable composition is flowable.

Preferably, the polymerizable composition polymerizes within 2 minutes to form the seal.

Preferably, the polymerizable composition comprises a silicone mixture.

Preferably, the silicone mixture comprises any one or more of the following components selected from the group comprising a siloxane polymer, a fumed amorphous silica, a platinum catalyst, a methyl hydrogen crosslinker, an inhibitor, a thixotropic agent optionally wherein the thixotropic agent is polydimethyl siloxane and an adhesion promoter optionally wherein the adhesion promoter is tetrapropoxysilane.

Preferably, the silicone mixture comprises:
(i) first silicone elastomer component comprising a first platinum catalyst; and
(ii) a second silicone elastomer component comprising a silicone crosslinker.

Preferably, the silicone mixture has a work time of between 30-90 seconds, and a cure time of between 1-5 minutes.

Preferably, prior to application of the polymerizable composition there is provided use of a base component in order to increase adhesion and/or improve cure time of the polymerizable composition. This can be in the form of a wipe. Ideally the base component comprises an adhesion promoter or a catalyst that accelerates polymerization of the polymerizable composition.

In another aspect of the invention there is provided an elastic seal comprising a polymerized composition, the seal being sized and shaped to naturally mimic contours and dimensions the head/neck or ear, the seal comprising:

(i) first silicone elastomer component comprising a first platinum catalyst; and
(ii) a second silicone elastomer component comprising a silicone crosslinker.

The seal is removable and is composed of the polymerized composition of the invention.

Also claimed is a kit for transiently sealing a head/neck or ear protective unit, comprising:

(i) a base component comprising at least one of an adhesion promoter or a catalyst that accelerates polymerization of the polymerizable composition and;
(ii) a polymerizable composition formulated to polymerize at the site of attachment to form a removable seal.

According to a further aspect of the invention there is provided a method of treatment of an inner ear injury, comprising:

applying to at least one of an auricle and an external ear canal a polymerizable composition;

wherein the polymerizable composition is formulated to cure to a hardness within 5 minutes; and

allowing the polymerizable composition to harden in the at least one of the auricle and external ear canal to the hardness, wherein the hardness is between 0 on the Shore 00 durometer scale and 40 on the Shore A durometer scale to so form an elastic protective barrier.

Preferably, the inner ear injury is selected from the group consisting of barotrauma, acoustic trauma, vestibular impairment, and noise induced hearing loss.

Preferably, the treatment is prophylactic treatment.

Preferably, during the step of applying, an electronic device is at least partially disposed in the at least one of the auricle and the external ear canal.

Preferably, the electronic device is an RFID chip, a hearing aid, or a communication device.

Preferably, the protective barrier is removable in a single piece and ideally has a tensile strength of between 200-800 psi and/or an elongation at break of between 400-650%.

Preferably, the polymerizable composition further comprises a pharmaceutical agent such as an analgesic, an antibiotic, an anti-inflammatory compound, and/or a steroid drug.

According to a yet further aspect of the invention there is provided a method of protecting an inner ear from barotrauma, acoustic trauma, vestibular impairment, and noise induced hearing loss, comprising:

applying to at least one of an auricle and an external ear canal a polymerizable composition;

wherein the polymerizable composition is formulated to cure to a hardness within 5 minutes; and

allowing the polymerizable composition to harden in the at least one of the auricle and external ear canal to the hardness, wherein the hardness is between 0 on the Shore 00 durometer scale and 40 on the Shore A durometer scale to so form an elastic protective barrier.

Preferably, the polymerizable composition comprises a silicon polymer ideally the silicon polymer comprises at least one of a siloxane polymer, a methyl hydrogen crosslinker, and a platinum catalyst, and optionally at least one of a fumed amorphous silica, a thixotropic agent, an adhesion promoter, and nano- or micron sized hollow spheres.

Preferably, the thixotropic agent comprises a polydimethyl siloxane and/or wherein the adhesion promoter is tetrapropoxysilane.

Preferably, the step of applying comprises a step of mixing a first and a second composition to form the polymerizable composition and more preferably the first composition comprises a catalyst and wherein the second composition comprises a crosslinker.

Preferably, the step of mixing is performed in a mixing nozzle or during the step of applying.

Preferably, the polymerizable composition is provided in a single-use dosage unit having a volume of between 1 ml and 5 ml.

Preferably, the polymerizable composition further comprises nano- or micron sized hollow spheres in an amount effective to further attenuate sound through the protective barrier.

All features ascribed to a particular invention are included mutatis mutandis to other aspects of the invention.

### Examples

Table 3 shows an exemplary two part polymerizable formulation having a dual adhesion promoter system. In this case, the two adhesion promoters work synergistically to increase adhesion to skin (or tissue) when compared to formulations having only one of the adhesion promoters. Without wishing to be bound by any particular theory, Applicant contemplates that one adhesion promoter makes the second more available at the surface of the formulation. Although the two adhesion promoters in this example are provided in Part 2 of the formulation, it should be appreciated that one adhesion promoter could be provided in each of Parts 1 and 2, that both adhesion promoters could be provided in Part 1, or that one adhesion promoter could be provided in the polymerizable formulations while a second adhesion promoter is provided in a base component.

The exemplary formulation of Table 3 is compounded to provide a short working and setting time. Specifically, the formulation has a working time of between 20-40 seconds (typically about 30 seconds), and a setting time of between 4-6 minutes (typically about 5 minutes) when Part 1 and Part 2 are mixed together and placed on skin.

TABLE 3

| Part 1 | | Part 2 | |
|---|---|---|---|
| Component | wt % | Component | wt % |
| Vinyl endblocked polydimethyl | 79.947 | Vinyl endblocked polydimethyl | 74-75 |
| siloxane polymer (chain length 350 | | siloxane polymer (chain length 350 | |
| repeating units) | | repeating units) | |
| Fumed silica with | 19.91 | Fumed silica with surface area of 200 m2/ | 18.5-19 |
| surface area of 200 m2/gram | | gram | |
| Platinum catalyst complex | 0.143 | Trimethyl endblocked methyl- | 3.5-4.0 |
| | | hydrogen siloxane polymer | |
| | | crosslinker (containing 50 wt % methyl | |
| | | hydrogen and 50 wt % dimethyl) | |
| | | 1,3,5,7-tetramethyl-1,3,5,7- | 0.013-0.017 |
| | | tetravinyl-cyclotetrasiloxane | |
| | | Tetrapropoxysilane adhesion | 2.2-2.8 |
| | | promoter | |
| | | N-(triethoxysilylpropyl)-0- | 0.37-0.45 |
| | | polyethylene oxide urethane | |
| | | adhesion promoter | |

It is also contemplated that the components shown in Table 1 could be included in Part 1 and Part 2 of the formulation in different concentration ranges as set forth below in the example of Table 4 achieving a desirable range of similar working times (e.g., between 10-120 seconds), similar setting times (e.g., between 1-10 minutes), similar adhesion properties (as described in Table 1), hardness of between 5-80 on the Shore A hardness scale, tensile strength between 200-1500 psi, and elasticities at break (of between 200-1000%).

TABLE 4

| Part 1 | | Part 2 | |
|---|---|---|---|
| Component | wt % | Component | wt % |
| Vinyl endblocked polydimethyl | 60-90 | Vinyl endblocked polydimethyl | 60-90 |
| siloxane polymer (100-1000 DP) | | siloxane polymer (100-1000 | |
| | | repeating siloxy units) | |
| Fumed silica with surface area of | 10-30 | Fumed silica with surface area of 200 m2/ | 10-30 |
| 200 m2/gram (100-400 m2/gram} | | gram | |
| Platinum catalyst complex | 0.06-0.2 | Trimethyl endblocked methyl- | 2-10 |
| | | hydrogen siloxane polymer | |
| | | crosslinker (containing 50% methyl | |
| | | hydrogen and 50% dimethyl) | |
| | | 1,3,5,7-tetramethyl-1,3,5,7- | .001-.05 |
| | | tetravinyl-cyclotetrasiloxane | |
| | | Tetrapropoxysilane adhesion | 1-5 |
| | | promoter | |
| | | N-(triethoxysilylpropyl)-0- | 0.1-2 |
| | | polyethylene oxide urethane | |
| | | adhesion promoter | |

Where specific additives are contemplated, it should be noted that these can be added in a simple manner to Part1, Part2, or both Parts as exemplarily shown in Table 5. In this exemplary formulation, barium sulfate is added as an imaging contrast agent, which is contemplated to have comparable work times (e.g., between 10-120 seconds), setting times (e.g., between 1-10 minutes), adhesion properties, hardness, tensile strength, and elasticities at break as the formulation of Table 1.

TABLE 5

| Part 1 | | Part 2 | |
|---|---|---|---|
| Component | wt % | Component | wt % |
| Vinyl endblocked polydimethyl | 66.6 ± 20 | Vinyl endblocked polydimethyl | 74.9 ± 20 |
| siloxane polymer | | siloxane polymer | |
| Fumed silica with | 16.7 ± 10 | Fumed silica with | 18.7 ± 10 |
| surface area of 200 m2/gram | | surface area of 200 m2/gram | |
| Platinum catalyst complex | 0.05 | Trimethyl endblocked methyl- | 3.7 ± 3 |
| | (0.001-0.2) | hydrogen siloxane polymer | |
| | | crosslinker (containing 50% methyl | |
| | | hydrogen and 50% dimethyl) | |
| Barium Sulfate (to make the | 16.7 ± 15 | 1,3,5,7-tetramethyl-1,3,5,7- | 0.015 |
| formulation radio-opaque) | | tetravinyl-cyclotetrasiloxane | (between |
| | | | 0.001-0.05 |
| | | Tetrapropoxysilane adhesion | 2.2 ± 2 |
| | | promoter | |
| | | N-(triethoxysilylpropyl)-0- | 0.37 (0.1-2) |
| | | polyethylene oxide urethane | |
| | | adhesion promoter | |

Table 6 shows yet another exemplary formulation including one or more pigments, which is contemplated to have comparable work times (e.g., between 10-90 seconds), setting times (e.g., between 1-10 minutes), adhesion properties, and elasticities at break as the formulation of Table 1.

TABLE 6

| Part 1 | | Part 2 | |
|---|---|---|---|
| Component | wt % | Component | wt % |
| Vinyl endblocked polydimethyl | 78.4 ± 20 | Vinyl endblocked polydimethyl | 74.9 ± 20 |
| siloxane polymer | | siloxane polymer | |
| Fumed silica with surface area of | 19.6 ± 10 | Fumed silica with surface area of | 18.7 ± 10 |
| 200 m2/gram | | 200 m2/gram | |
| Platinum catalyst complex | 0.06 | Trimethyl endblocked methyl- | 3.7 ± 3 |
| | (0.001-0.2) | hydrogen siloxane polymer | |
| | | crosslinker (containing 50% methyl | |
| | | hydrogen and 50% dimethyl) | |
| Pigment (e.g., yellow, | 1.96 ± 1.5 | 1,3,5,7-tetramethyl-1, | 0.015 |
| orange, green, blue, brown) | | 3,5,7-tetravinyl-cyclo-tetrasiloxane | (between |
| | | | 0.001-0.05) |
| | | Tetrapropoxy silane adhesion | 2.2 ± 2 |
| | | promoter | |
| | | N-(triethoxysilylpropyl)-0- | 0.37 |
| | | polyethylene oxide urethane | (0.1-2) |
| | | adhesion | |
| | | promoter | |

Tables 7-10 show further exemplary formulations only including one adhesion promoter, which is contemplated to have comparable work times (e.g., between 10-90 seconds), setting times (e.g., between 1-10 minutes), hardness, tensile strength, and elasticities at break as the formulation of Table 1, but a lower adhesion strength to skin likely due to a lack of synergistic effect with a second adhesion promoter.

TABLE 7

| Part 1 | | Part 2 | |
|---|---|---|---|
| Component | wt % | Component | wt % |
| Vinyl endblocked polydimethyl | 79.95 ± 20 | Vinyl endblocked polydimethyl siloxane | 75.29 ± 20 |
| siloxane polymer | | polymer | |
| Fumed silica with surface area of | 19.99 ± 10 | Fumed silica with surface area of 200 m2/ | 18.8 ± 10 |
| 200 m2/gram | | gram | |
| Platinum catalyst complex | 0.06 | Trimethyl endblocked methyl-hydrogen | 3.8 ± 3 |
| | (0.001-0.2) | siloxane polymer crosslinker (containing | |
| | | 50% methyl hydrogen and 50% dimethyl) | |
| | | 1,3,5,7-tetramethyl-1,3,5,7-tetravinyl- | 0.015 |
| | | cyclotetrasiloxane | (between |
| | | | 0.001-0.05) |
| | | Tetrapropoxysilane adhesion promoter | 2.3 ± 2 |

TABLE 8

| Part 1 | | Part 2 | |
|---|---|---|---|
| Component | wt % | Component | wt % |
| Vinyl endblocked | 79.95 ± 20 | Vinyl endblocked polydimethyl siloxane | 75.29 ± 20 |
| polydimethyl siloxane | | polymer | |
| polymer | | | |
| Fumed silica with surface | 19.99 ± 10 | Fumed silica with surface area of 200 m2/ | 18.8 ± 10 |
| area of 200 m2/gram | | gram | |
| Platinum catalyst complex | 0.06 | Trimethyl endblocked methyl-hydrogen | 3.8 ± 3 |
| | (0.001-0.2) | siloxane polymer crosslinker (containing | |
| | | 50% methyl hydrogen and 50% dimethyl) | |
| | | 1,3,5,7-tetramethyl-1,3,5,7- | 0.015 |
| | | tetravinyl-cyclotetrasiloxane | (betwe |
| | | | 0.001-0.05) |
| | | N-(triethoxysilylpropyl)-0- | 0.37 |
| | | polyethylene oxide urethane adhesion | (0.1-2) |
| | | promoter | |

TABLE 9

| Part 1 | | Part 2 | |
|---|---|---|---|
| Component | wt % | Component | wt % |
| Vinyl endblocked polydimethyl | 79.95 ± 20 | Vinyl endblocked polydimethyl | 75.29 ± 20 |
| siloxane polymer | | siloxane polymer | |
| Fumed silica with surface area of | 19.99 ± 10 | Fumed silica with surface area of | 18.8 ± 10 |
| 200 m2/gram | | 200 m2/gram | |
| Platinum catalyst complex | 0.06 | Trimethyl endblocked methyl- | 3.8 ± 3 |
| | (0.001-0.2) | hydrogen siloxane polymer | |
| | | crosslinker (containing 50% | |
| | | 1,3,5,7-tetramethyl-1,3,5,7- | 0.015 |
| | | tetravinyl-cyclotetrasiloxane | (between |
| | | | 0.001-0.05) |
| | | Tetrapropoxysilane adhesion | 2.57 ± 2.5 |
| | | promoter | |

TABLE 10

| Part 1 | | Part 2 | |
|---|---|---|---|
| Component | wt % | Component | wt % |
| Vinyl endblocked polydimethyl | 79.95 ± 20 | Vinyl endblocked polydimethyl | 75.29 ± 20 |
| siloxane polymer | | siloxane polymer | |
| Fumed silica with surface area of | 19.99 ± 10 | Fumed silica with surface area of | 18.8 ± 10 |
| 200 m2/gram | | 200 m2/gram | |
| Platinum catalyst complex | 0.06 | Trimethyl endblocked methyl- | 3.8 ± 3 |
| | (0.001-0.2) | hydrogen siloxane polymer | |
| | | crosslinker (containing 50% methyl | |
| | | hydrogen and 50% dimethyl) | |
| | | 1,3,5,7- | 0.015 |
| | | tetramethyl- | (between |
| | | 1,3,5,7-tetravinyl- | 0.001-0.05) |
| | | cyclotetra- | |
| | | siloxane | |
| | | N-(triethoxysilylpropyl)- | 2.57 ± 2.5 |
| | | 0-polyethylene oxide | |
| | | urethane adhesion | |
| | | promoter | |

Table 11 shows a further exemplary formulation that includes a thixotropic agent added at a concentration of between 0.25-3 wt % to make the formulation non-slump. The formulation of Table 11 is contemplated to have comparable work times (e.g., between 10-90 seconds), setting times (e.g., between 1-10 minutes), adhesion properties, hardness, tensile strength, and elasticities at break as the formulation of Table 1.

TABLE 11

| Part 1 | | Part 2 | |
|---|---|---|---|
| Component | wt % | Component | wt % |
| Vinyl endblocked polydimethyl | 79.32 ± 20 | Vinyl endblocked polydimethyl | 74.9 ± 20 |
| siloxane polymer | | siloxane polymer | |
| Fumed silica with surface area | 19.83 ± 10 | Fumed silica with surface area of | 18.7 ± 10 |
| of 200 m2/gram | | 200 m2/gram | |
| Platinum catalyst complex | 0.06 | Trimethyl endblocked methyl- | 3.7 ± 3 |
| | (0.001-0.2) | hydrogen siloxane polymer | |
| | | crosslinker (containing 50% | |
| | | methyl hydrogen and 50% | |
| | | dimethyl) | |
| Hydroxyl endblocked | 0.79 | 1,3,5,7-tetramethyl- | 0.015 |
| polydimethyl siloxane | (between | 1,3,5,7-tetravinyl- | (between |
| (thixotrope) | 0.25-3) | cyclotetrasiloxane | 0.001-0.05) |
| | | Tetrapropoxysilane adhesion | 2.2 ± 2 |
| | | promoter | |
| | | N- | 0.37 |
| | | (triethoxysilylpropyl)- | (0.1-2) |
| | | 0-polyethylene | |
| | | oxide urethane | |
| | | adhesion promoter | |

Table 12 shows yet another exemplary formulation including less platinum catalyst than the formulation of Table 1, which is contemplated to require a longer cure time.

TABLE 12

| Part 1 | | Part 2 | |
|---|---|---|---|
| Component | wt % | Component | wt % |
| Vinyl endblocked polydimethyl | 79.97 ± 20 | Vinyl endblocked polydimethyl | 74.9 ± 20 |
| siloxane polymer | | siloxane polymer | |
| Fumed silica with surface area of | 19.99 ± 10 | Fumed silica with surface area of | 18.7 ± 10 |
| 200 m2/gram | | 200 m2/gram | |
| Platinum catalyst complex | 0.04 | Trimethyl endblocked methyl- | 3.7 ± 3 |
| | (0.001-0.2) | hydrogen siloxane polymer | |
| | | crosslinker (containing 50% methyl | |
| | | hydrogen and 50% dimethyl) | |
| | | 1,3,5,7-tetramethyl-1,3,5,7- | 0.015 |
| | | tetravinyl-cyclotetrasiloxane | (between |
| | | | 0.001-0.05) |
| | | Tetrapropoxysilane | 2.2 ± 2 |
| | | adhesion | |
| | | N-(triethoxysilylpropyl)- | 0.37 |
| | | 0-polyethylene oxide | (0.1-2) |
| | | urethane adhesion | |
| | | promoter | |

Table 13 shows further exemplary formulation including less crosslinker than the formulation of Table 1, which is contemplated to require a longer cure time.

TABLE 13

| Part 1 | | Part 2 | |
|---|---|---|---|
| Component | wt % | Component | wt % |
| Vinyl endblocked polydimethyl | 79.95 ± 20 | Vinyl endblocked polydimethyl | 76 ± 20 |
| siloxane polymer | | siloxane polymer | |
| Fumed silica with surface area of | 19.997 ± 10 | Fumed silica with surface area of | 79 ± 10 |
| 200 m2/gram | | 200 m2/gram | |
| Platinum catalyst complex | 0.06 | Trimethyl endblocked methyl- | 2.3 ± 2 |
| | (0.001-0.2) | hydrogen siloxane polymer | |
| | | crosslinker (containing 50% methyl | |
| | | hydrogen and 50% dimethyl) | |
| | | 1,3,5,7- | 0.015 |
| | | tetramethyl-1,3,5,7- | (between |
| | | tetravinyl-cyclotetrasil oxane | 0.001-0.05) |
| | | Tetrapropoxysilane adhesion | 2.3 ± 2 |
| | | promoter | |
| | | N-(triethoxysilylpropyl)- | 0.38 |
| | | 0-polyethylene oxide | (0.1-2) |
| | | urethane adhesion | |
| | | promoter | |

Table 14 shows another exemplary formulation including no fumed silica.

TABLE 14

| Part 1 | | Part 2 | |
|---|---|---|---|
| Component | wt % | Component | wt % |
| Vinyl endblocked polydimethyl | 99.93 | Vinyl endblocked polydimethyl | 92.1 ± 20 |
| siloxane polymer | | siloxane polymer | |
| Platinum catalyst complex | 0.07 | Trimethyl endblocked methyl- | 4.6 ± 4 |
| | (0.001-0.2) | hydrogen siloxane polymer | |
| | | crosslinker (containing 50% methyl | |
| | | hydrogen and 50% dimethyl) | |
| | | 1,3,5,7-tetramethyl- | 0.018 |
| | | 1,3,5,7-tetravinyl- | (between |
| | | cyclotetrasiloxane | 0.001-0.05) |
| | | Tetrapropoxysilane adhesion | 2.8 ± 2 |
| | | promoter | |
| | | N-(triethoxysilylpropyl)-0- | 0.46 (0.1-2) |
| | | polyethylene oxide urethane | |
| | | adhesion promoter | |

As discussed above, contemplated seals may be formed to maintain a piece of equipment on skin of a user such that the equipment need not be retained with straps, tapes, or other manners of affixing. In other words, contemplated seals will act as a temporary adhesive that affixes the equipment to skin of a user. For example, contemplated equipment includes sensors (e.g., thermal sensors such as body temperature sensors, optical sensors such as blood oxygenation sensors, electrical sensors such as electrolyte sensors, magnetic and/or motion sensors, positional sensors such as GPS sensors, etc.), which will typically be coupled to the trunk or extremity of a user. Other suitable equipment also includes communication devices such as mono- and bidirectional in-ear radios that can be retained in the outer ear canal, as well as passive equipment such as sunglasses, wires, etc.

Advantageously, the adhesive seal between the skin and the equipment can be formed from flowable materials as described herein to sealingly conform to a user's body geometry while also releasably bonding to the equipment. Within short time, the material will polymerize to the flexible seal that now strongly adheres to the user.

Reference herein to “substrate” is intended to refer to the surface of a head/neck or ear-piece to which the compositions of the invention may be applied.

Reference herein to “periphery of an apparatus” is intended to refer the edge or contact area/zone of the head/neck or ear-piece apparatus which is to be sealed to the skin of the appropriate area of an individual.

Reference herein to a “head/neck or earpiece” is intended to refer to any form of head protective apparatus with the specific purpose of preventing auditory damage to an individual.

Reference herein to a “combatant” is intended to refer to an individual involved in a battle/war scenario, a hunter, a gun enthusiast or anyone else that may use gun or other equipment that has a loud sound emission.

Reference herein to “an individual” is intended to refer not only to a combatant but also to an individual who is exposed to loud noises in a working environment such as road and construction workers, airport personnel, runway personnel and any other profession which involves use of noisy machinery and have the potential to suffer from auditory damage such as and including attendees of rock concerts, artists and audience alike.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1.-40. (canceled)

41. A method of sealing a substrate surface of a protective apparatus or a communication apparatus in situ to skin of a head, neck, or ear region of an individual, comprising:
placing a polymerizable composition between the protective apparatus or the communication apparatus and the skin of the individual and allowing the polymerizable composition to polymerize to form a seal in situ,
wherein the seal is removable and formed in situ to sealingly conform to a contour of a specific region of skin to which the substrate surface of the protective or communication apparatus is applied.

42. The method according to claim 41 wherein the polymerizable composition polymerizes within 2 minutes of contact with the skin and/or substrate surface of the protective or communication apparatus.

43. The method of claim 41 wherein the polymerizable composition comprises a silicone mixture.

44. The method of claim 43 wherein the silicone mixture comprises any one or more of the following components selected from the group comprising a siloxane polymer, a fumed amorphous silica, a platinum catalyst, a methyl hydrogen crosslinker, an inhibitor, a thixotropic agent optionally wherein the thixotropic agent is polydimethyl siloxane and an adhesion promoter optionally wherein the adhesion promoter is tetrapropoxysilane.

45. The method of claim 43 wherein the silicone mixture comprises:
(i) a first silicone elastomer component comprising a first platinum catalyst; and
(ii) a second silicone elastomer component comprising a silicone crosslinker.

46. The method of claim 41 wherein the silcone mixture has a work time of between 30-90 seconds, and a cure time of between 1-5 minutes.

47. The method of claim 41 wherein the seal has a hardness of between 0 on the Shore 00 durometer scale and 40 on the Shore A durometer scale.

48. The method of claim 41 wherein the seal has a hardness of between 15-25 on the Shore A durometer scale.

50. The method of claim 41 wherein the communication apparatus is a hearing aid, or an ear piece of a communication device.

51. A method of treatment of an inner ear injury, comprising:
applying to at least one of an auricle and an external ear canal a polymerizable composition;
wherein the polymerizable composition is formulated to cure to a hardness within 5 minutes; and
allowing the polymerizable composition to harden in the at least one of the auricle and external ear canal to the hardness, wherein the hardness is between 0 on the Shore 00 durometer scale and 40 on the Shore A durometer scale to so form an elastic protective barrier.

52. The method of claim 51 wherein the inner ear injury is selected from the group consisting of barotrauma, acoustic trauma, vestibular impairment, and noise induced hearing loss.

53. The method of claim 51 wherein the treatment is prophylactic treatment.

54. The method of claim 51 wherein the polymerizable composition comprises at least one of a siloxane polymer, a methyl hydrogen crosslinker, and a platinum catalyst, and optionally at least one of a fumed amorphous silica, a thixotropic agent, an adhesion promoter, and nano- or micron sized hollow spheres, or wherein the thixotropic agent comprises a polydimethyl siloxane, and/or wherein the adhesion promoter is tetrapropoxysilane.

55. The method of claim 51 wherein, during the step of applying, an electronic device is at least partially disposed in the at least one of the auricle and the external ear canal.

56. The method of claim 51 wherein the protective barrier is removable in a single piece.

57. The method of claim 51 wherein the polymerizable composition further comprises a pharmaceutical agent.

58. A method of protecting an inner ear from barotrauma, acoustic trauma, vestibular impairment, and noise induced hearing loss, comprising:
applying to at least one of an auricle and an external ear canal a polymerizable composition;
wherein the polymerizable composition is formulated to cure to a hardness within 5 minutes; and
allowing the polymerizable composition to harden in the at least one of the auricle and external ear canal to the hardness, wherein the hardness is between 0 on the Shore 00 durometer scale and 40 on the Shore A durometer scale to so form an elastic protective barrier.

59. The method of claim 58 wherein the polymerizable composition comprises a silicon polymer.

60. The method of claim 59 wherein the silicon polymer comprises at least one of a siloxane polymer, a methyl hydrogen crosslinker, and a platinum catalyst, and optionally at least one of a fumed amorphous silica, a thixotropic agent, an adhesion promoter, and nano- or micron sized hollow spheres.
